(19) United States
(12) Patent Application Publication
(10) Pub. No.: US 2022/0378593 A1
(43) Pub. Date: Dec. 1, 2022

(54) STENT DELIVERY SYSTEM, ENDOSCOPE SYSTEM, AND STENT INDWELLING METHOD

(71) Applicant: OLYMPUS CORPORATION, Tokyo (JP)

(72) Inventor: Yoichi SAKURADA, Higashimurayama-shi (JP)

(73) Assignee: OLYMPUS CORPORATION, Tokyo (JP)

(21) Appl. No.: 17/882,700

(22) Filed: Aug. 8, 2022

Related U.S. Application Data
(63) Continuation of application No. PCT/JP2020/006775, filed on Feb. 20, 2020.

(51) Int. Cl. A61F 2/95; A61F 2/966

(52) U.S. Cl. CPC A61F 2/9522; A61F 2/966

(57) ABSTRACT

A stent delivery system includes: a first outer tube; a second outer tube disposed at a proximal side from the first outer tube; an inner tube connected to a distal end part of the second outer tube and configured to be inserted into the first outer tube; a traction member connected to a proximal end part of the first outer tube and configured to be inserted into the second outer tube; and a stent housed between the inner tube and the first outer tube. The surface roughness of an outer surface of the second outer tube is higher than the surface roughness of an outer surface of the first outer tube.

The present disclosure relates to a stent delivery system, an endoscope system, and a stent indwelling method. This application is a continuation application based on International Patent Application No. PCT/JP2020/006775 filed on Feb. 20, 2020, the contents of the PCT international application is incorporated herein by reference.

### BACKGROUND

A procedure of indwelling a stent to expand stenosis or obstruction (hereinafter referred to as “stenosis, etc.”) in the gastrointestinal tract is known. A stent delivery system is used to indwell a stent in a stenosis or the like. The stent delivery system transports a stent to a stenosis or the like through a treatment tool channel of an endoscope.

For example, in a conventional stent delivery system disclosed in Published Japanese Translation No. 2007-526096 of the PCT International Publication, a guide wire tube inserted through an inside of the sheath is provided so as to be slidable with respect to the sheath. The stent is housed in a gap between the guidewire tube and the sheath on a distal end part of the delivery system. The stent stored on the distal end part is indwelled in a stenosis or the like by pulling the sheath with respect to the guide wire tube toward a proximal side.

An operation of pulling the sheath with respect to the guide wire tube toward the proximal side is performed by an assistant who assists the endoscopist who operates the endoscope.

The assistant pulls the sheath with respect to the guide wire tube toward the proximal side while fixing the guide wire tube so that the position of the guide wire tube does not move, thereby the stent is indwelled in the target position.

In conventional stent delivery systems, when the sheath is retracted to the proximal side, for example, a guide wire tube moves to the distal side due to the reaction generated by the contact between the curved sheath and the treatment tool channel of the endoscope. In this case, a stent is indwelled in a position deviated from the target position. In order to suppress the occurrence of this phenomenon, a coordinated operation in which the endoscopist adjusts the position of the stent delivery system in accordance with the operation of pulling the sheath by the assistant is performed.

### SUMMARY

A first aspect of the disclosure relates to a stent delivery system including: a first outer tube; a second outer tube disposed at a proximal side from the first outer tube; an inner tube connected to a distal end part of the second outer tube and configured to be inserted into the first outer tube; a traction member connected to a proximal end part of the first outer tube and configured to be inserted into the second outer tube; and a stent housed between the inner tube and the first outer tube. The surface roughness of an outer surface of the second outer tube is higher than the surface roughness of an outer surface of the first outer tube.

A second aspect of the present disclosure relates to an endoscope system including a stent delivery system including: a first outer tube, a second outer tube disposed at a proximal side from the first outer tube, an inner tube connected to a distal end part of the second outer tube and configured to be inserted into the first outer tube, a traction member connected to a proximal end part of the first outer tube and configured to be inserted into the second outer tube, and a stent housed between the inner tube and the first outer tube, and an endoscope having a channel through which the stent delivery system is insertable. The surface roughness of an outer surface of the second outer tube is higher than the surface roughness of an outer surface of the first outer tube.

A third aspect of the present disclosure relates to a stent indwelling method using an endoscope and a stent delivery device including steps of: inserting the stent delivery system into a channel of the endoscope; inserting a distal end of the stent delivery system toward an indwelling position of a stent through the channel; fixing the endoscope and a second outer tube after positioning the distal end of the stent delivery system at the indwelling position, the second outer tube being disposed at a proximal side from a first outer tube of the stent delivery system and having an outer surface in which the surface roughness of the outer surface is higher than the surface roughness of an outer surface of the first outer tube; tracting the first outer tube toward a proximal side by a traction member connected to a proximal end part of the first outer tube and configured to be inserted into the second outer tube to expose a stent housed between the inner tube and the first outer tube from the first outer tube.

### BRIEF DESCRIPTION OF THE DRAWINGS

FIG. 1 is a view illustrating the overall configuration of an endoscope system according to the first Embodiment.

FIG. 2 is a view illustrating the stent delivery system of the endoscope system.

FIG. 3 is a cross-sectional and a partial interrupted view illustrating the stent delivery system.

FIG. 4 is a view illustrating the overall configuration of the inner tube, the second outer tube, and a chip of the stent delivery system.

FIG. 5 is a view illustrating the stent delivery system inserted into a treatment instrument channel of the endoscope of the endoscope system.

FIG. 6 is a view describing the operation of an endoscopist when indwelling a stent into a target position.

FIG. 7 is a view illustrating the stent delivery system when a stent is indwelled.

FIG. 8 is a cross-sectional and a partial interrupted view illustrating the stent delivery system of the endoscope system according to the second embodiment.

FIG. 9 is a view describing the operation of an endoscopist when indwelling a stent into a target position.

FIG. 10 is a cross-sectional and a partial interrupted view illustrating the stent delivery system of the endoscope system according to the third embodiment.

FIG. 11 is a cross-sectional and a partial interrupted view illustrating the stent delivery system of the endoscope system according to the fourth embodiment.

FIG. 12 is a cross-sectional and a partial interrupted view illustrating the curved stent delivery system.

FIG. 13 is a view illustrating the stent delivery system when a stent is indwelled.

FIG. 14 is a view illustrating the stent delivery system when a stent is indwelled.

FIG. 15 is a cross-sectional view illustrating the stent delivery system in which a housing position of a stent is fixed in by using an elevator.

FIG. 16 is a view illustrating the endoscope of a modified example of the embodiment.

FIG. 17 is a view illustrating the endoscope of a modified example of the embodiment.

FIG. 18 is a view illustrating the second outer tube of a modified example of the embodiment.

### DETAILED DESCRIPTION OF EXEMPLARY EMBODIMENTS

### First Embodiment

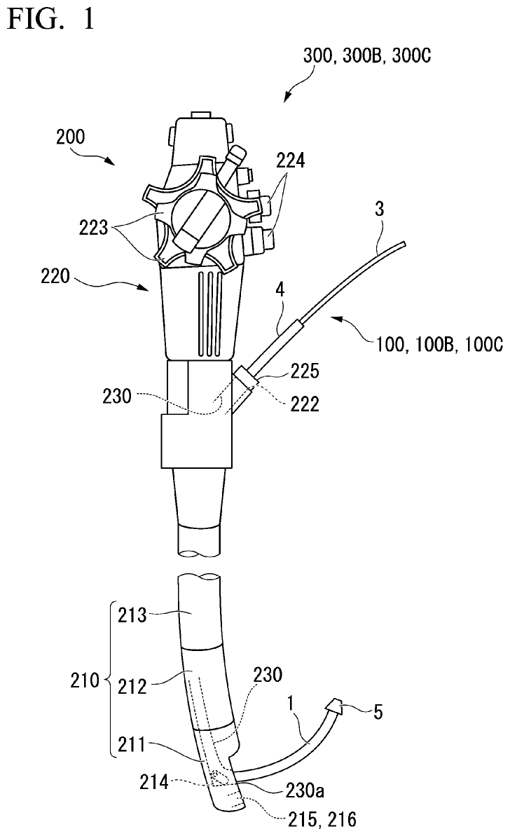

The first embodiment of the present disclosure will be described with reference to FIGS. 1 to 7.

[Endoscope System 300]

FIG. 1 is a view illustrating the overall configuration of an endoscope system 300 according to the first Embodiment. The endoscope system 300 includes an endoscope 200 and a stent delivery system 100 inserted into the channel of the endoscope 200.

[Endoscope 200]

The endoscope 200 is a known side-view type flexible endoscope. The endoscope 200 has an elongated insertion portion 210 and an operation portion 220 provided at a proximal end portion of the insertion portion 210. The endoscope 200 may use a direct-view type flexible endoscope.

The insertion portion 210 includes a distal end hard portion 211 provided at the distal end portion thereof, a curved portion 212 attached to a proximal end side of the distal end hard portion 211 and configured to be bent by an operation, and a flexible tube portion 213 attached to a proximal end side of the curved portion 212. An image pickup unit 216 having a light guide 215 and a CCD is provided on a side surface of the distal end hard portion 211 in a state exposing to an outside.

A treatment tool channel 230 for inserting an endoscopic treatment tool such as a stent delivery system 100 is formed in the insertion portion 210. A distal end part 230a of the treatment tool channel 230 is opened on a side surface of the distal end hard portion 211. A proximal end part of the treatment tool channel 230 extends to the operation portion 220.

An elevator 214 is provided on the distal end hard portion 211 of the treatment tool channel 230. A proximal end portion of the elevator 214 is rotatably supported by the distal end hard portion 211. An elevator operation wire (not shown) fixed to a tip end portion of the elevator 214 extends through the insertion portion 210 toward the proximal side.

The curved portion 212 is configured to be capable of being curved in the left-right direction and the upside-down direction. A distal end of the operation wire is fixed to a distal end side of the curved portion 212. The operation wire extends through the insertion portion 210 to the operation portion 220.

A knob 223 for operating the operation wire, a switch 224 for operating the image pickup unit 216, or the like are provided on a proximal end side of the operation portion 220. The user is capable of bending the curved portion 212 in a desired direction by operating the knob 223.

A forceps port 222 communicating with the treatment tool channel 230 is provided on a distal end side of the operation portion 220. The user can insert an endoscopic treatment tool such as the stent delivery system 100 from the forceps port 222. A forceps plug 225 preventing body fluid from leaking is attached to the forceps port 222.

[Stent Delivery System 100]

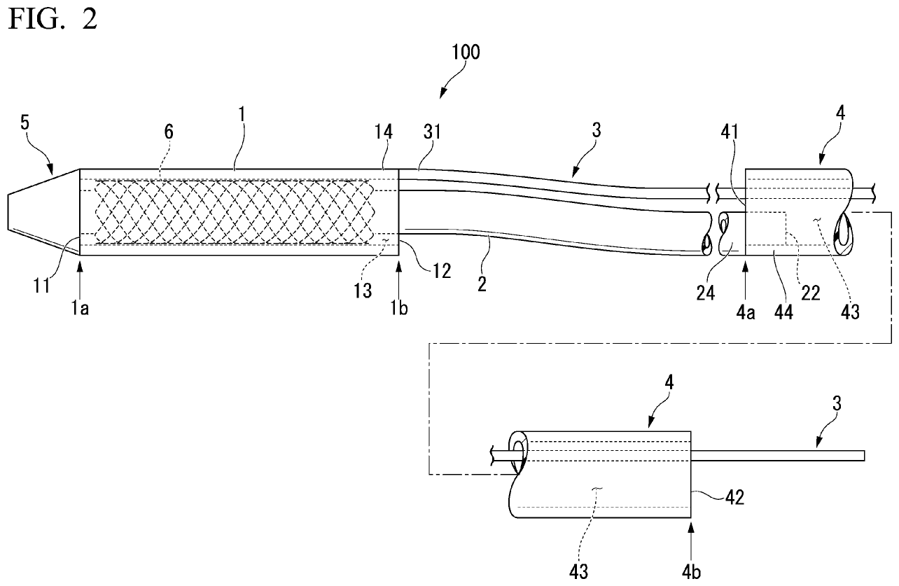

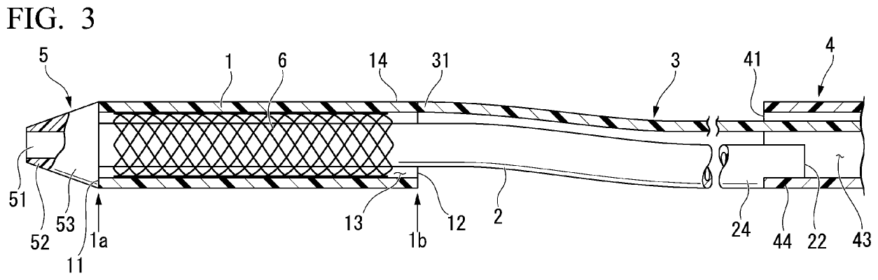

FIG. 2 is a view illustrating the stent delivery system 100. FIG. 3 is a cross-sectional and a partial interrupted view illustrating the stent delivery system. The stent delivery system 100 is formed in an elongated shape as a whole. The stent delivery system 100 includes a first outer tube 1, an inner tube 2, a traction member 3, a second outer tube 4, a tip 5, and a stent 6.

The first outer tube 1 is an elongated tubular member insertable into the treatment tool channel 230 of the endoscope 200. The first outer tube 1 is made of resin or the like and has flexibility. In the first outer tube 1, a first opening 11 is opened at a distal end 1a and a second opening 12 is opened at a proximal end 1b. The first opening 11 and the second opening 12 communicate with the internal space (lumen) 13 of the first outer tube 1. The first opening 11 and the second opening 12 are substantially circular openings through which the inner tube 2 is insertable.

The traction member 3 is an elongated member insertable into the treatment tool channel 230 of the endoscope 200. The traction member 3 is made of resin or the like and has flexibility. A distal end portion 31 of the traction member 3 is connected to a proximal end portion 14 of the first outer tube 1. The user can pull the first outer tube 1 toward the proximal end side by pulling the traction member 3.

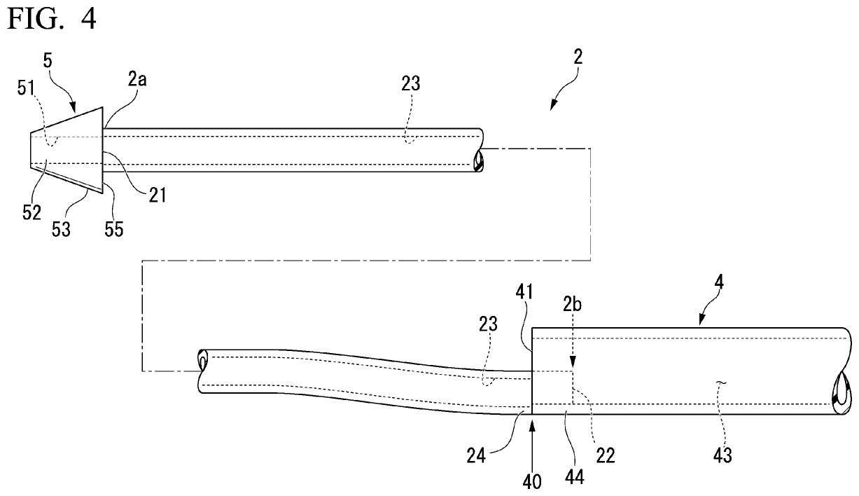

FIG. 4 a view illustrating the overall configuration of the inner tube 2, the second outer tube 4, and the chip 5. The inner tube 2 is an elongated tubular member insertable into the treatment tool channel 230 of the endoscope 200. The inner tube 2 is made of resin or the like and has flexibility. In the inner tube 2, a distal end opening 21 is opened at a distal end 2a and a proximal opening 22 is opened at a proximal end 2b. The distal end opening 21 and the proximal opening 22 communicate with the lumen (guide wire lumen) 23 of the inner tube 2.

As shown in FIG. 2, the inner tube 2 passes through the first opening 11 and the second opening 12 and is inserted into the lumen 13 of the first outer tube 1 so as to be relatively movable. The outer diameter of the inner tube 2 inserted through the lumen 13 of the first outer tube 1 is smaller than the inner diameter of the lumen 13 of the first outer tube 1.

As shown in FIG. 2, the second outer tube 4 is an elongated tubular member insertable into the treatment tool channel 230 of the endoscope 200. The second outer tube 4 is made of resin or the like and has flexibility. In the second outer tube 4, a first opening 41 is opened at the distal end 4a and a second opening 42 is opened at the proximal end 4b. The first opening 41 and the second opening 42 communicate with the internal space (lumen) 43 of the second outer tube 4. The first opening 41 and the second opening 42 are substantially circular openings through which the traction member 3 is insertable.

As shown in FIG. 4, a distal end portion 44 of the second outer tube 4 is connected to a proximal end portion 24 of the inner tube 2. The internal space (lumen) 43 of the second outer tube 4 communicates with the lumen (guide wire lumen) 23 of the inner tube 2. The guide wire G inserted into the lumen (guide wire lumen) 23 of the inner tube 2 extends to the proximal side through the internal space (lumen) 43 of the second outer tube 4.

As shown in FIG. 2, the traction member 3 passes through the first opening 41 and the second opening 42 and is inserted into the internal space (lumen) 43 of the second outer tube 4 so as to be relatively movable. The outer diameter of the traction member 3 inserted through the internal space (lumen) 43 of the second outer tube 4 is smaller than the inner diameter of the lumen 13 of the second outer tube 4.

As shown in FIG. 4, the chip 5 has a substantially conical shape and has a through hole 51 extending in the axial direction. The tip 5 has a distal end 52 and a proximal end 53 and is connected to the inner tube 2 on the proximal end 53 side. The distal end has a diameter smaller than a diameter of the proximal end 53. Since the diameter of the proximal end 53 is larger than the outer diameter of the inner tube 2, there is a step 55 at a connection portion between the tip 5 and the inner tube 2. Since the through hole 51 communicates with the lumen 23 of the inner tube 2 via the distal end opening 21, the guide wire G is entered into the guide wire lumen of the inner tube 2 when the guide wire G is inserted into the through hole 51 of the tip 5.

The stent 6 is a tubular self-expanding stent. The stent 6 is formed by weaving a wire. The stent 6 is housed in a gap between the inner tube 2 and the first outer tube 1 in a state where the inner tube 2 is passed through the inside of the stent 6 and the diameter of the stent is reduced. The stent 6 is locked to a locking portion (not shown) formed on the outer peripheral surface of the inner tube 2. Accordingly, the stent 6 is positioned with respect to the inner tube 2 in the reduced diameter state, and does not move relative to the inner tube 2 in the longitudinal direction. The stent 6 may use a laser-cut type stent formed by cutting a metal cylinder by a laser.

The materials of the first outer tube 1 and the inner tube 2 are not particularly limited as long as the desired mechanical properties are satisfied in the first outer tube 1 and the inner tube 2. As the material of the first outer tube 1, the inner tube 2, the traction member 3, and the second outer tube 4, the following can be exemplified. Olefin resins such as polypropylene and polyethylene, and their copolymer resins, polyester resins such as polyethylene terephthalate (PET) and polybutylene terephthalate (PBT), and general-purpose resins such as polyvinyl alcohol (PVA). Engineering resins such as polyamide resins, fluororesins (e.g, polytetrafluoroethylene (PTFE), polyvinylidene fluoride (PVDF), PFA, FEP, ETFE, etc.), polyetheretherketone (PEEK, etc.). Various elastomer resins (polystyrene-based, polyolefin-based, polyurethane-based, polyester-based, polyamide-based, polyvinyl chloride-based, etc.), silicone-containing resins, polyurethane-based resins, etc. may be used alone or in combination. Further, in order to suppress the occurrence of buckling and the like, a material composite with a mesh made of stainless steel or the like may also be used. For stent delivery systems used under X-ray fluoroscopy, X-ray opaque metallic markers (medical X-ray opaque metals and alloys such as platinum, tungsten, and iridium) may be added or X-ray opaque, and various materials (such as barium sulfate) may be mixed.

The wire forming the stent 6 is a superelastic alloy containing NiTi as the main material. The superelastic alloy containing NiTi as the main material is not permanently deformed at the time of weaving, and the weaving shape is memorized by applying heat treatment in the weaved state.

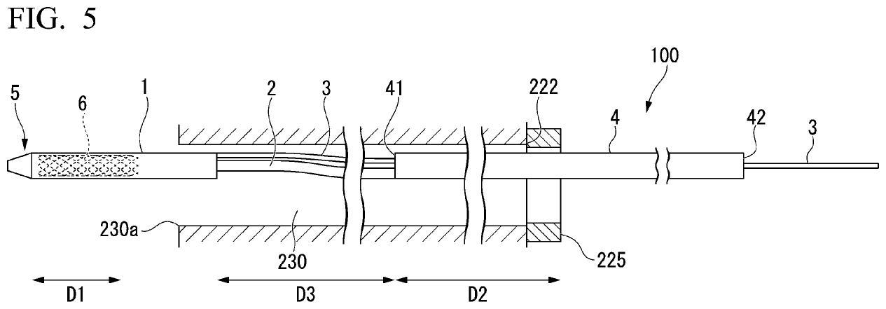

FIG. 5 is a view illustrating the stent delivery system 100 inserted into the treatment instrument channel 230. The distal end of the stent delivery system 100 shown in FIG. 5 projects from a distal end 230a of the treatment tool channel 230 of the endoscope 200.

In a state where the stent 6 storage location of the stent delivery system 100 is projected from the distal end 230a of the treatment tool channel 230 of the endoscope 200 and the stent 6 is located at the indwelling position, the second opening 12 of the first outer tube 1 and the first opening 41 of the second outer tube 4 are located inside the treatment tool channel 230. The second opening 42 of the second outer tube 4 is located outside the treatment tool channel 230.

In FIG. 5, the inner tube 2 and the traction member 3 are parallel to each other in the intermediate portion between the first outer tube 1 and the second outer tube 4. A longitudinal length D3 of the intermediate portion is longer than a longitudinal length D1 of the stent 6.

A stent indwelling method using the endoscope system 300 including the stent delivery system 100 configured as described above will be described with an exemplary example of a procedure for placing a stent 6 in a bile duct.

The endoscopist inserts the insertion portion 210 of the endoscope 200 into the body cavity of the patient through a natural opening such as the mouth. At that time, the endoscopist operates the knob 223 or the like to bend the curved portion 212 as necessary.

The endoscopist passes the guide wire G through the treatment tool channel 230 of the endoscope 200, and inserts the guide wire G into the bile duct while observing with the endoscope 200. Subsequently, the endoscopist operates the guide wire G under fluoroscopy to pass through the stenosis site in the bile duct and move the distal end of the guide wire G closer to the liver than the stenosis site (target position).

The endoscopist inserts the proximal end of the guide wire G protruding from the forceps plug 225 of the endoscope 200 into the through hole 51 of the tip 5 of the stent delivery system 100. The guide wire G enters the guide wire lumen 23 of the inner tube 2 through the through hole 51.

The endoscopist advances the stent delivery system 100 along the guide wire G by pushing the stent delivery system 100 while holding the guide wire G (insertion step). The distal end of the stent delivery system 100 projects from the the distal end 230a of the treatment tool channel 230 of the endoscope 200. When the distal end of the stent delivery system 100 passes through the stenosis site (target position), the endoscopist advances and retreats the stent delivery system 100 to determine the indwelling position of the stent 6. The endoscopist may insert the stent delivery system 100 into the treatment tool channel 230 without using the guide wire G.

As shown in FIG. 5, in a state where the stent 6 storage location of the stent delivery system 100 is projected from the distal end 230a of the treatment tool channel 230 of the endoscope 200 and the stent 6 is located in the indwelling position, the first opening 41 of the second outer tube 4 is located inside the treatment tool channel 230. In the state, the second outer tube 4 is extended from the forceps plug 225. The traction member 3 is extended from the second outer tube 4 outside the treatment tool channel 230.

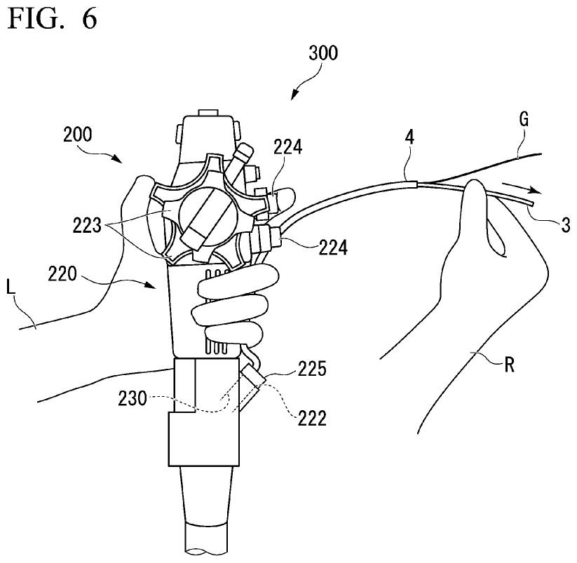

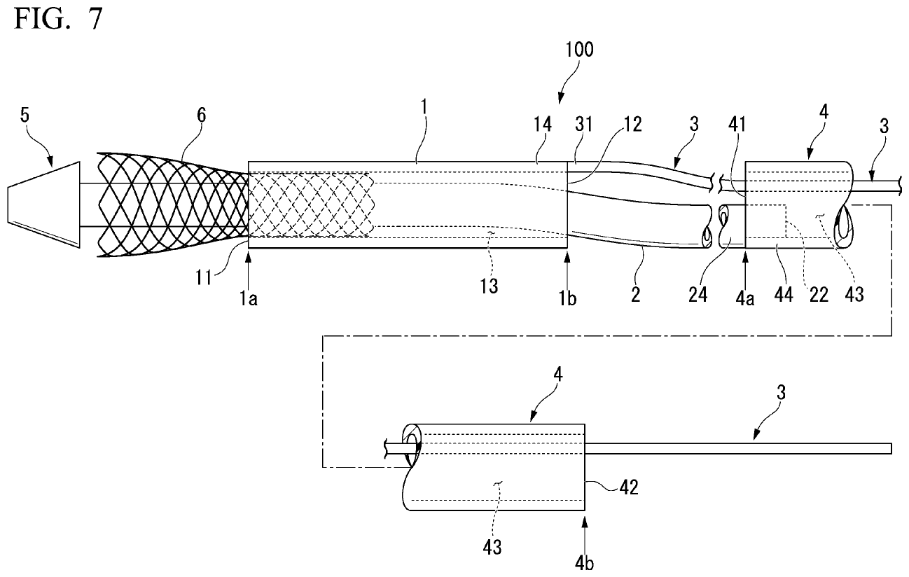

FIG. 6 is a view describing the operation of an endoscopist when indwelling the stent 6 into a target position. FIG. 7 is a view illustrating the stent delivery system when a stent 6 is indwelled. After determining the target position of the stent 6, as shown in FIG. 6, the endoscopist pulls the traction member 3 toward the proximal side with the hand R (traction step) while fixing the second outer tube 4 to the vicinity of the forceps plug 225 of the operation portion 220 with the other hand L which holding the operation portion 220 of the endoscope 200 (fixing step). Then, the first outer tube 1 retracts with respect to the inner tube 2. As a result, as shown in FIG. 7, the stent 6 is gradually exposed from the distal end side thereof and expands. The endoscopist can perform the procedure of indwelling the stent 6 while operating the endoscope 200 without the assistance of an assistant.

The endoscopist pulls the traction member 3 toward the proximal side while fixing the second outer tube 4 to the operation portion 220, so that only the outer tube 1 retracts to the proximal side without retracting and advancing the inner tube 2 with respect to the endoscope channel 230. Therefore, the position of the inner tube 2 is maintained, and the position in which the stent 6 is housed is unlikely to deviate from the target position.

The stent placement operation may be performed by an endoscopist and an assistant. The endoscopist manually fixes a position of the second outer tube 4 in the vicinity of the forceps plug 225. In that state, the assistant pulls the traction member 3 toward the proximal side with one hand while lightly holding the proximal end side of the second outer tube 4 with the other hand. Then, the stent is easily indwelled to the target position without the necessity of a coordinated operation of the endoscopist with the assistant.

The longitudinal length D3 of the intermediate portion is, as shown in FIG. 5, longer than the longitudinal length D1 of the stent 6. Therefore, even when the traction member 3 is pulled toward the proximal side until the stent 6 is indwelled, the first outer tube 1 and the second outer tube 4 do not come into contact with each other.

When the stent 6 is completely exposed, the stent 6 expands radially at the entire area of the axial direction, and the inner diameter of the stent 6 becomes larger than the outer diameter of the inner tube 2. Accordingly, the lock between the stent 6 and the inner tube 2 is also released.

In a state before the stent 6 is completely expanded, the stent 6 can be housed again (recapture) between the first outer tube 1 and the inner tube 2 by advancing the first outer tube 1 with respect to the inner tube 2 and reduces the diameter of the stent 6. Recapture is useful when resetting the indwelling position.

When the endoscopist retracts the inner tube 2 after the locking between the stent 6 and the inner tube 2 is released, the stent 6 stays in the indwelling position and the inner tube 2 is removed from the stent 6.

When the endoscopist pulls out the stent delivery system 100 excluding the stent 6, the indwelling procedure of the stent 6 is completed. Then, a contrast tube may be introduced along the guide wire G and a contrast agent may be used to confirm the open state of the stenosis.

According to the stent indwelling method using the endoscope system 300 including the stent delivery system 100 according to the present embodiment, the endoscopist can easily indwell the stent 6 at a target position such as stenosis without the assistance of the assistant. The endoscopist can retract only the traction member 3 and the first outer tube 1 toward the proximal side by fixing the second outer tube 4 to the operation portion 220. Therefore, the position of the inner tube 2 is maintained, and the position in which the stent 6 is housed is unlikely to deviate from the target position.

Although the first embodiment has been described in detail with reference to the drawings, the specific configuration is not limited to the embodiment and includes design changes and the like within a range not deviating from the gist of the present invention. The components shown in the above-described embodiment and the modifications shown below can be appropriately combined and configured.

### Second Embodiment

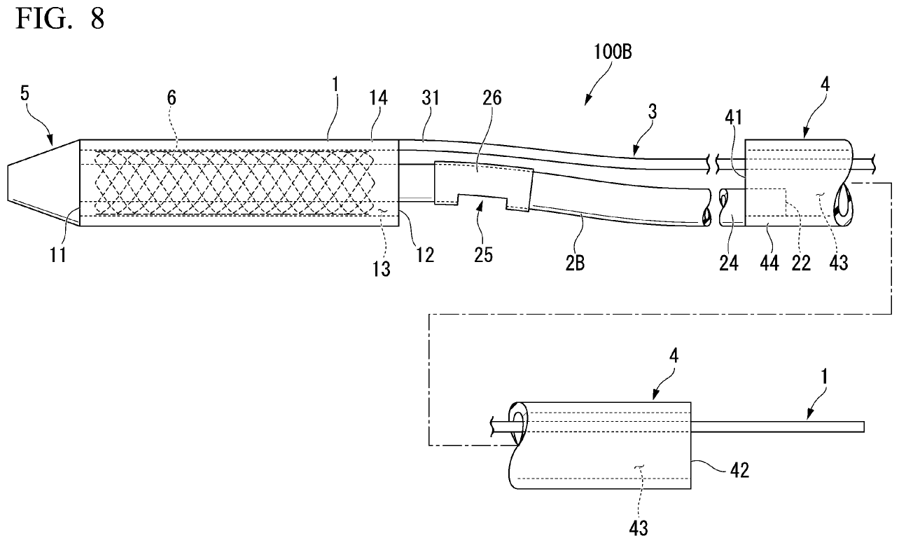

The second embodiment of the present disclosure will be described with reference to FIG. 8. In the following description, the same constituent elements as those already described in the first embodiment are designated by the same reference signs, and duplicate descriptions thereof will be omitted. The endoscope system 300B according to the second embodiment is different from the endoscope system 300 according to the first embodiment in that the inner tube has an opening on a side portion.

The endoscope system 300B includes the endoscope 200 and the stent delivery system 100B inserted into the channel of the endoscope 200.

FIG. 8 is a cross-sectional and a partial interrupted view illustrating the stent delivery system of the endoscope system 100B. The stent delivery system 100B includes a first outer tube 1, an inner tube 2B, a traction member 3, a second outer tube 4, a tip 5, and a stent 6.

The inner tube 2B has the same configuration as the inner tube 2 of the first embodiment, and further includes an opening 25. The opening 25 is formed on the side portion of the inner tube 2B and communicates with the lumen (guide wire lumen) 23 of the inner tube 2. The opening 25 is an opening through which a guide wire is insertable.

As shown in FIG. 8, the opening 25 is located in an intermediate portion between the first outer tube 1 and the second outer tube 4 in a state where the stent 6 is housed.

In order to prevent buckling and the like from occurring in the opening 25, as shown in FIG. 8, a reinforcing member 26 is attached to the peripheral portion of the opening 25. The reinforcing member 26 is a member wound in the circumferential direction along a surface of the inner tube 2 at an outer periphery around the opening 25. The material of the reinforcing member 26 is preferably, but is not limited to, a material composited with a mesh formed of a resin or stainless steel which have resistance to buckling or the like.

The guide wire G inserted through the through hole 51 of the tip 5 passes through the opening 25 and is pulled out of the stent delivery system 100B. By pulling out the guide wire G from the intermediate portion of the stent delivery system 100B, the guide wire G can be shortened, and the stent delivery system 100 B is easily introduced into the treatment tool channel 230.

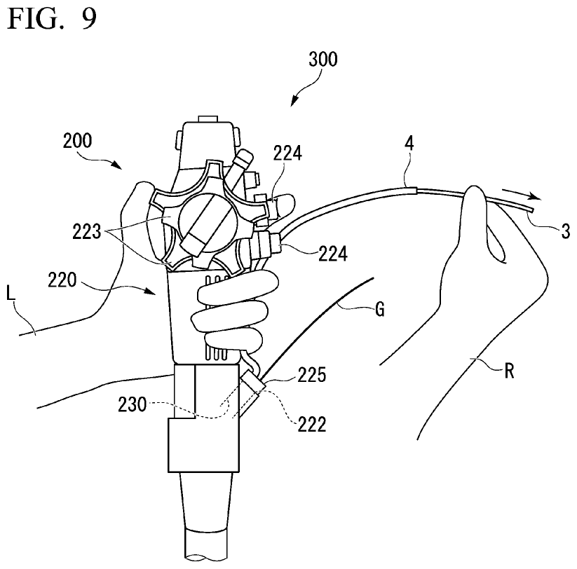

FIG. 9 is a view describing the operation of an endoscopist when indwelling the stent 6 into the target position. The guide wire G is pulled out from the forceps plug 225 separately from the second outer tube 4. Since the guide wire G is not inserted through the second outer tube 4, the endoscopist easily operates the traction member 3 and the second outer tube 4.

According to the stent indwelling method using the endoscope system 300B including the stent delivery system 100B according to the present embodiment, the endoscopist easily indwells the stent 6 at a target position such as stenosis without the assistance of an assistant. The position of the inner tube 2B can be maintained, and the position where the stent 6 is housed is unlikely to deviate from the target position.

Although the second embodiment of the present disclosure has been described in detail with reference to the drawings, the specific configuration is not limited to this embodiment and includes design changes and the like within a range not deviating from the gist of the present invention. The components shown in the above-described embodiments and modifications can be appropriately combined and configured.

### Third Embodiment

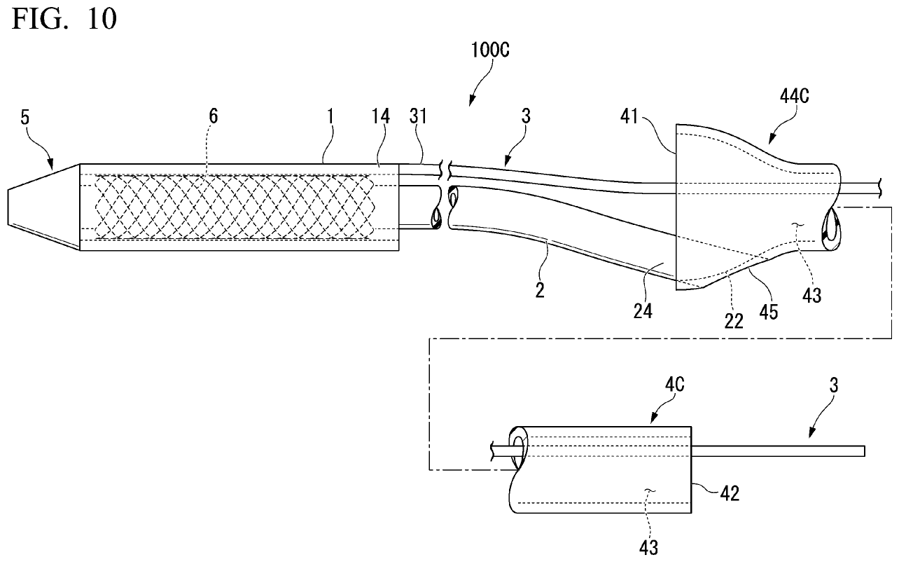

The third embodiment of the present disclosure will be described with reference to FIG. 10. In the following description, the same constituent elements as those already described in the first embodiment are designated by the same reference signs, and duplicate description will be omitted. The endoscope system 300C according to the second embodiment is different from the endoscope system 300 according to the first embodiment in that the second outer tube has an opening.

The endoscope system 300C includes the endoscope 200 and the stent delivery system 100C inserted through the channel of the endoscope 200.

FIG. 10 is a cross-sectional and a partial interrupted view illustrating the stent delivery system of the endoscope system 100C. The stent delivery system 100C includes the first outer tube 1, the inner tube 2, the traction member 3, the second outer tube 4C, the tip 5, and the stent 6.

The second outer tube 4C is, as shown in FIG. 10, an elongated tubular member insertable into the treatment tool channel 230 of the endoscope 200. The second outer tube 4 is made of resin or the like and has flexibility. In the second outer tube 4, a first opening 41 is opened at the distal end 4a and a second opening 42 is opened at the proximal end 4b. The first opening 41 and the second opening 42 communicate with the internal space (lumen) 43 of the second outer tube 4. The first opening 41 and the second opening 42 are substantially circular openings through which the traction member 3 is insertable.

The tip portion 44C of the second outer tube 4C is formed in a flare shape having a large outer diameter on the distal end side. The flare shape is a shape such that the diameter is gradually spread toward one end and formed like bell shape. An opening 45 is formed on the side of the distal end portion 44C of the second outer tube 4C. The opening 45 communicates with the lumen (guide wire lumen) 23 via the proximal end opening 22 of the inner tube 2. The opening 45 is an opening through which a guide wire is insertable.

The distal end portion 44C of the second outer tube 4C is connected to the proximal end portion 24 of the inner tube 2. The lumen (guide wire lumen) 23 of the inner tube 2 passes through the first opening 41 and is connected to the opening 45. The guide wire G inserted into the lumen (guide wire lumen) 23 of the inner tube 2 is pulled out of the stent delivery system 100C through the opening 45. By pulling out the guide wire G from the intermediate portion of the stent delivery system 100C, the guide wire G can be shortened, and the stent delivery system 100C is easily introduced into the treatment tool channel 230.

The opening 45 is formed on the side portion of the tip portion 44C formed in a flare shape. Therefore, when the guide wire G is pulled out to the outside of the stent delivery system 100C, the guide wire and the second outer tube 4C are less likely to come into contact with each other, and the reaction force generated by the contact is less likely to occur.

According to the stent indwelling method using the endoscope system 300C including the stent delivery system 100C according to the present embodiment, the endoscopist easily indwells the stent 6 at a target position such as stenosis without the assistance of the assistant. The position of the inner tube 2 can be maintained, and the position where the stent 6 is housed is unlikely to deviate from the target position.

Although the third embodiment of the present disclosure has been described in detail with reference to the drawings, the specific configuration is not limited to this embodiment and includes design changes and the like within a range not deviating from the gist of the present invention. The components shown in the above-described embodiments and modifications can be appropriately combined and configured.

### Fourth Embodiment

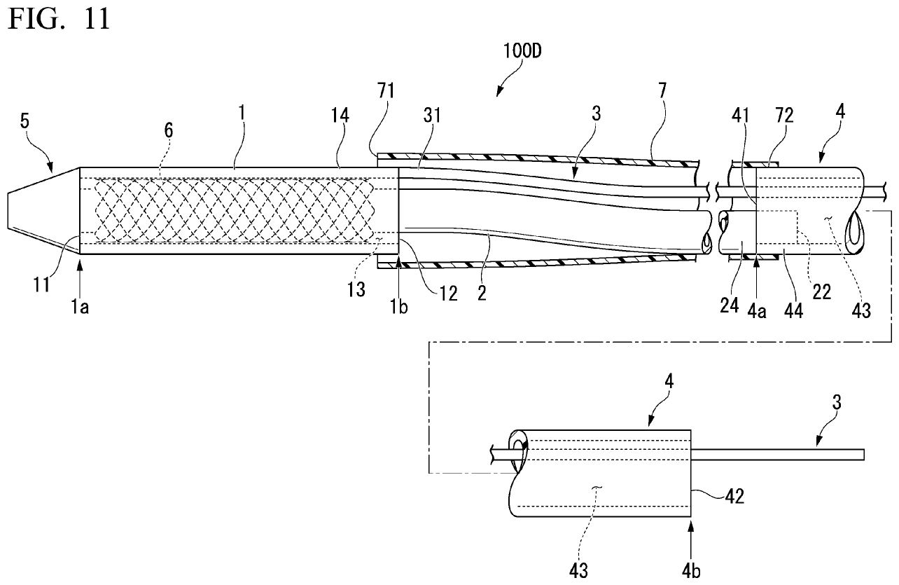

The fourth embodiment of the present disclosure will be described with reference to FIGS. 11 to 14. In the following description, the same constituent elements as those already described in the first embodiment are designated by the same reference signs, and duplicate description will be omitted. The endoscope system 300D according to the fourth embodiment is different from the endoscope system 300 according to the first embodiment in that an intermediate sheath 7 is provided.

The endoscope system 300D includes the endoscope 200 and the stent delivery system 100D inserted into the channel of the endoscope 200.

FIG. 11 is a cross-sectional and a partial interrupted view illustrating the stent delivery system 100D. The stent delivery system 100D includes the first outer tube 1, the inner tube 2, the traction member 3, the second outer tube 4, the tip 5, the stent 6, and an intermediate sheath 7.

The intermediate sheath 7 is an elongated tubular member insertable into the treatment tool channel 230 of the endoscope 200. The intermediate sheath 7 is made of an easily slipped material (fluorine-based resin or the like) and has flexibility. In the intermediate sheath 7, a first opening 71 is opened at the distal end and a second opening 72 is opened at the proximal end. The second opening 72 is fixed to the outer peripheral surface of the distal end 4a of the second outer tube 4. The inner tube 2 and the traction member 3 are inserted into the internal space of the intermediate portion sheath 7.

The longitudinal length of the intermediate sheath 7 is longer than the longitudinal length D3 of the intermediate. Therefore, in a state where the intermediate portion sheath 7 is not curved, the first opening 71 is located on the outer peripheral side of the proximal end portion 14 of the first outer tube, and is located on the proximal side from the most proximal end side of the stent 6. The first opening 71 is not fixed to the proximal end portion 14 of the first outer tube 1. In the intermediate portion sheath 7, the first opening 71 may be fixed to the proximal end portion 14 of the first outer tube, and the second opening 72 may not be fixed to the distal end 4a of the second outer tube 4.

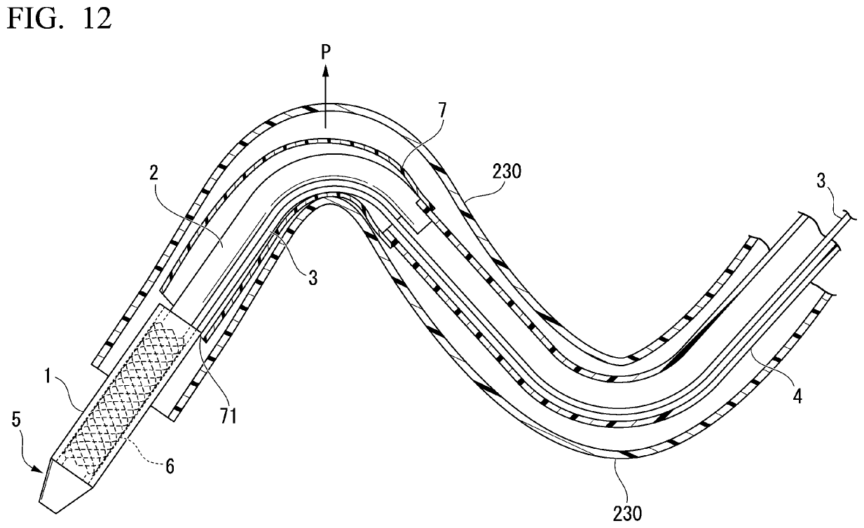

FIG. 12 is a cross-sectional and a partial interrupted view illustrating the curved stent delivery system 100D. If the intermediate portion is curved when the traction member 3 is pulled, the inner tube 2 in the intermediate portion may bend in the bending direction P. The stent delivery system 100D has an intermediate portion sheath 7 so as to cover the intermediate portion, and the inner tube 2 comes into contact with the inner peripheral surface of the intermediate portion sheath 7. Accordingly, it is possible to suppress bending the inner tube 2 in the bending direction P.

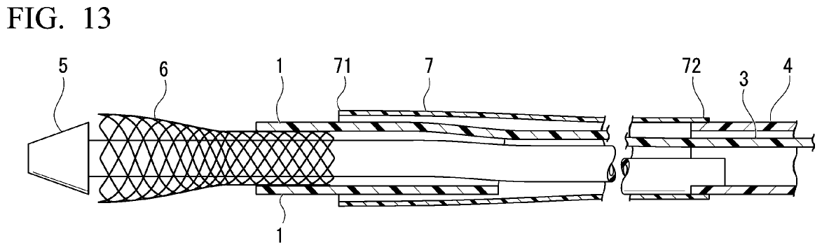

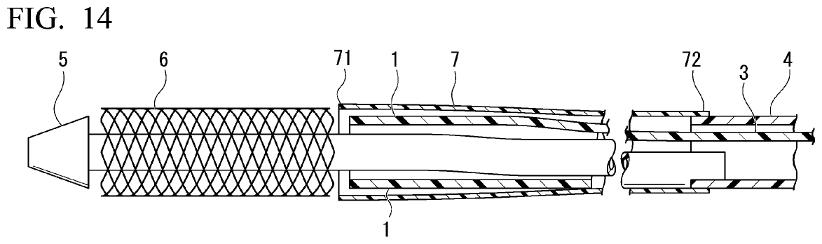

FIGS. 13 and 14 are views illustrating the stent delivery system 100D when a stent 6 is indwelled. When the traction member 3 is pulled to indwell the stent 6, the first opening 71 of the intermediate sheath 7 is located on the outer peripheral side of the proximal end 14 of the first outer tube 1 and is located on the proximal side from the most proximal end side of the stent 6 regardless of the curved shape of the intermediate sheath 7. Therefore, the stent delivery system 100D can indwell the stent 6 without the occurrence of hooking the stent on the intermediate sheath 7.

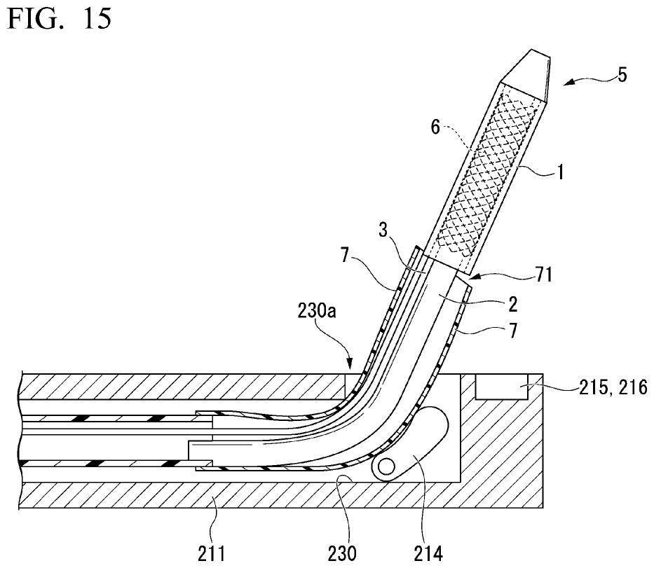

FIG. 15 is a cross-sectional view illustrating the stent delivery system 100D in which a housing position of a stent 6 is fixed by using the elevator 214. When indwelling the stent 6, the endoscopist may rotate the elevator 214 to sandwich the intermediate sheath 7 between the elevator 214 and the treatment tool channel 230. The endoscopist to indwell the stent 6 by pulling the traction member 3 toward the proximal side by using one hand R, while fixing the second outer tube 4 to the vicinity of the forceps plug 225 of the operation portion 220 with the other hand L. Since the intermediate portion sheath 7 is sandwiched between the elevator 214 and the like, the distal end position of the first outer tube 1 and the housed position of the stent 6 are unlikely to deviate. As a result, when the stent 6 is indwelled, the deviation of the placement position is unlikely to occur.

According to the stent indwelling method using the endoscope system 300D including the stent delivery system 100D according to the present embodiment, the endoscopist easily indwells the stent 6 at a target position such as stenosis without the assistance of the assistant. The inner tube 2 is less likely to bend in the bending direction P, and the position where the stent 6 is housed is less likely to deviate from the target position. The position where the stent 6 is housed is unlikely to deviate from the target position by fixing the intermediate sheath 7 using the elevator 214.

### Modified Example 1

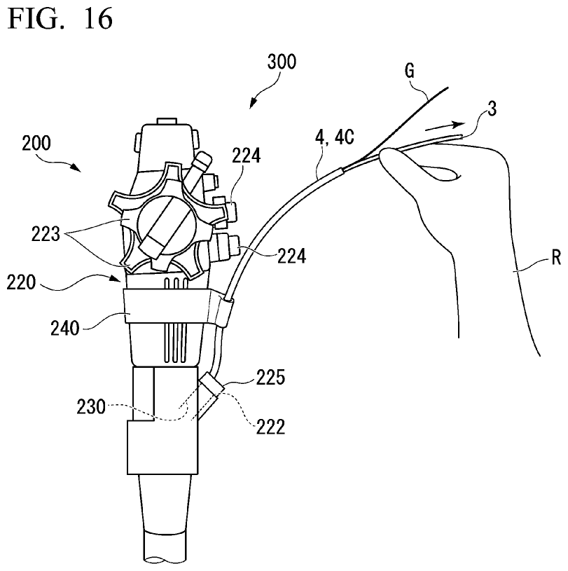

For example, in the above embodiment, although the endoscopist holds the second outer tube 4 in the vicinity of the forceps plug 225 of the operation portion 220 by using one hand L holding the operation portion 220 of the endoscope 200, the fixing mode of the inner tube 2 is not limited to this. As shown in FIG. 16, the endoscope 200 may further include a fixing member 240. The fixing member 240 is attached in the vicinity of the operation portion 220, and the second outer tube 4 can be fixed so that the relative positions of the inner tube 2 and the operation portion 220 do not change. Due to usage of the fixing member 240, the endoscopist does not need to fix the second outer tubes 4, 4C with one hand L, and can concentrate on the operation of the traction member 3.

### Modified Example 2

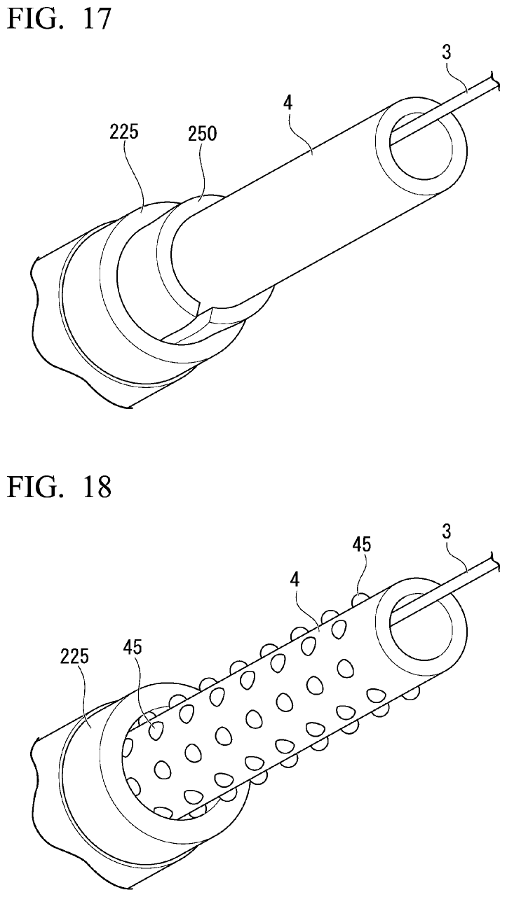

As shown in FIG. 17, the endoscope 200 may further have an insertion member 250. The second outer tube 4 may be fixed to the endoscope 200 by sandwiching the insertion member (fixing member) 250 between the second outer tube 4 and the forceps plug 225. The insertion member 250 is, for example, formed in a cylindrical shape having a cut in the longitudinal direction. The insertion member 250 has flexibility and is sandwiched between the second outer tube 4 and the forceps plug 225. The insertion member 250 is made of a resin having a high frictional resistance in order to fix the relative position between the second outer tube 4 and the forceps plug 225. The insertion member 250 is not limited to the cylindrical shape member, and may be any member that can be sandwiched between the forceps plug 225 and the second outer tube 4.

### Modified Example 3

The fixing of the second outer tube 4 to the endoscope 200 may be performed by increasing the reaction of the second outer tube 4 to the forceps plug 225. In order to increase the reaction force, as shown in FIG. 18, for example, the outer peripheral surface 4S of the second outer tube 4 may have a surface with a large surface roughness such as an uneven shape. The outer peripheral surface 4S of the second outer tube 4 has a higher surface roughness than the surface roughness of the outer peripheral surface of the first outer tube 1. The surface roughness may be imparted by mechanically or chemically processing the second outer tube 4. A method may be used in which the surface roughness is imparted by attaching a member having a surface roughness around the second outer tube.

### Modified Example 4

For example, in the above embodiment, the stent 6 is a self-expandable stent, but the stent is not limited to the self-expandable stent. The stent may be a non-self-expandable stent, and examples thereof include a CoCr-based alloy stent and a biodegradable stent made of polylactic acid, polyglycolic acid, and a copolymer thereof. Further, the stent may be a stent that expands with a fluid. For example, the stent may be a non-self-expandable stent that is expanded by another treatment tool such as a balloon.

Although the second embodiment of the present invention has been described in detail with reference to the drawings hereinbefore, a specific configuration is not limited to the embodiment, and includes design changes without departing from the gist of the present invention. In addition, it is possible to combine and configure components shown in the embodiment described above and a modified example as appropriate.

What is claimed is:

1. A stent delivery system comprising:
a first outer tube;
a second outer tube disposed at a proximal side from the first outer tube;
an inner tube connected to a distal end part of the second outer tube and configured to be inserted into the first outer tube;
a traction member connected to a proximal end part of the first outer tube and configured to be inserted into the second outer tube; and
a stent housed between the inner tube and the first outer tube,
wherein a surface roughness of an outer surface of the second outer tube is higher than a surface roughness of an outer surface of the first outer tube.

2. The stent delivery system according to claim 1, wherein the inner tube and the traction member are parallel to each other in an intermediate portion between the first outer tube and the second outer tube.

3. The stent delivery system according to claim 2, wherein a length of the intermediate portion in a longitudinal direction is longer than a length of the stent in the longitudinal direction.

4. The stent delivery system according to claim 1, wherein
the inner tube has a guide wire lumen through which a guide wire is insertable, and
an opening communicating with the guide wire lumen is formed in the inner tube.

5. The stent delivery system according to claim 2, wherein
the inner tube has a guide wire lumen through which a guide wire is insertable, and
an opening communicating with the guide wire lumen is formed in the inner tube at a position of the intermediate portion.

6. The stent delivery system according to claim 1, wherein
the inner tube has a guide wire lumen through which a guide wire is insertable, and
an opening communicating with the guide wire lumen is formed in the second outer tube.

7. The stent delivery system according to claim 6, wherein
a distal end opening of the second outer tube is formed in a flare shape.

8. An endoscope system comprising:
a stent delivery system comprising
a first outer tube,
a second outer tube disposed at a proximal side from the first outer tube,
an inner tube connected to a distal end part of the second outer tube and configured to be inserted into the first outer tube,
a traction member connected to a proximal end part of the first outer tube and configured to be inserted into the second outer tube, and
a stent housed between the inner tube and the first outer tube; and
an endoscope having a channel through which the stent delivery system is insertable,
wherein a surface roughness of an outer surface of the second outer tube is higher than a surface roughness of an outer surface of the first outer tube.

9. The endoscope system according to claim 8, wherein the endoscope has a fixing member configured to fix the inner tube.

10. The endoscope system according to claim 9, wherein the fixing member is an insertion member sandwiched between a forceps plug of the endoscope and the second outer tube.

11. The endoscope system according to claim 8, wherein
the inner tube has a guide wire lumen through which a guide wire is insertable, and
an opening communicating with the guide wire lumen is formed in the second outer tube.

12. The endoscope system according to claim 11, wherein
a distal end opening of the second outer tube is formed in a flare shape.

13. A stent indwelling method using an endoscope and a stent delivery device comprising steps of:
inserting the stent delivery system into a channel of the endoscope;
inserting a distal end of the stent delivery system toward an indwelling position of a stent through the channel;
fixing the endoscope and a second outer tube after positioning the distal end of the stent delivery system at the indwelling position, the second outer tube being disposed at a proximal side from a first outer tube of the stent delivery system and having an outer surface in which a surface roughness of the outer surface is higher than a surface roughness of an outer surface of the first outer tube;
tracting the first outer tube toward a proximal side by a traction member connected to a proximal end part of the first outer tube and configured to be inserted into the second outer tube to expose a stent housed between the inner tube and the first outer tube from the first outer tube.

14. The stent indwelling method according to claim 13, wherein
the second outer tube is fixed to a vicinity of a forceps plug of an operation part of the endoscope when fixing the second outer tube to the endoscope.

15. The stent indwelling method according to claim 13, wherein
the distal end of the stent delivery system is inserted into the channel to a target position by using a guide wire when fixing the second outer tube with the endoscope.
